(19) United States
(12) Patent Application Publication
(10) Pub. No.: US 2008/0195221 A1
(43) Pub. Date: Aug. 14, 2008

(54) IMPLANT AND A METHOD FOR PARTIAL REPLACEMENT OF JOINT SURFACES

(75) Inventors: Ralph Howald, Gossau (CH); Markus Frohlich, Balterswil (CH); Christian Bruderer, Winterthur (CH)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER GMBH, WINTERTHUR (CH)

(21) Appl. No.: 12/017,754

(22) Filed: Jan. 22, 2008

(30) Foreign Application Priority Data
Jan. 22, 2007 (EP) EP07100948

(51) Int. Cl. A61F 2/32; A61B 17/56

(52) U.S. Cl. 623/22.32; 623/22.31; 606/301

(57) ABSTRACT

An orthopedic implant for the partial replacement of joint surfaces. In one exemplary embodiment, the orthopedic implant includes an articular portion having a concave articular surface and a base body. The orthopedic implant may be sized to replace a small portion of the patient's natural acetabulum. In this manner, the orthopedic implant allows for the repair and/or replacement of a small portion of the articular cartilage of the acetabulum. In one exemplary embodiment, a plurality of different sized orthopedic implants may be connected together to replace a larger portion articular cartilage.

### CROSS-REFERENCE TO RELATED APPLICATIONS

This application is based upon and claims priority to European Application Serial No. EP07100948, entitled “An implant and a method for partial replacement of joint surfaces”, filed on Jan. 22, 2007, the entire disclosure of which is expressly incorporated by reference herein.

### BACKGROUND

1. Field of the Invention

The present invention relates to orthopedic components and methods of implanting the same.

2. Background of the Related Art

Degenerative and traumatic damage to the articular cartilage of skeletal joints can result in pain and restricted motion. During the early stages of articular cartilage degeneration in the hip joint, the articular cartilage at the edge of the acetabulum may degrade and begin to undergo delamination. As indicated above, in its early stages, this damage is present at the edges of the acetabulum where the articulation strain on the cartilage is comparatively low. Thus, the damage in these areas may not result from loading stress, but, instead, may arise due to the neck of the femur striking the edge of the acetabulum during joint articulation. While such contact is undesirable, in specific bone morphologies, this contact results in constantly reoccurring contusions of the articular cartilage at specific points on the acetabulum, causing cartilage damage and, potentially, delamination.

### SUMMARY

The present invention provides an orthopedic implant for the partial replacement of joint surfaces. In one exemplary embodiment, the orthopedic implant includes an articulation portion having a concave articular surface and a base body. The orthopedic implant may be sized to replace a portion of the patient's natural acetabulum. In this manner, the orthopedic implant allows for the repair and/or replacement of a portion of the articular cartilage of the acetabulum. In one exemplary embodiment, a plurality of differently sized orthopedic implants may be connected together to replace a portion of the acetabulum.

In one exemplary embodiment, the articular surface of the orthopedic component has a substantially spheroidal shape that approximates the interior of a portion or section of a sphere. Stated another way, the articular surface has a concave substantially spherical surface. The articulation portion defining the articular surface is generally made from cartilage-compatible substances on which the natural cartilage can articulate without undergoing a substantial rate of wear. In exemplary embodiments, the articulation portion may be formed from hydrogels, polyethylenes (PE), polyurethanes (PU), polycarbonate urethanes (PCU), polyetheretherketone (PEEK), polyetherketoneketone (PEKK), poly ethylene-co-vinyl alcohol (EVAL), polyvinyl alcohol (PVA), or materials of pyrolytic carbon. The cartilage-compatible materials may include a fiber-reinforcement and/or have a woven or non-woven structure or scaffold. A scaffold-like design may be advantageous to allow additional invasion of chondrocytes and its precursor cells in order to form new cartilage on top of the cartilage compatible materials In one exemplary embodiment, the articulation portion is formed from a cartilage-compatible material that is arranged on the base body. In one exemplary embodiment, the base body is at least partially formed from a highly porous biomaterial. In an embodiment, the implant can be flexible to allow geometrical adaptation of the implant to the defect morphology.

In one exemplary embodiment, the base body of the orthopedic implant provides support to the articulation portion and is sized to allow the base body to be received within the acetabulum in a pelvic bone. In this manner, the base body provides for the securement of the orthopedic implant in the acetabulum. In one exemplary embodiment, the base body includes a plurality of anchorage members extending therefrom that may facilitate attachment to the acetabulum by providing for securement in the areas of greatest bone stock of the pelvis.

Advantageously, the present invention allows for the replacement of the damaged cartilage of the acetabulum without otherwise influencing the remaining joint surface. Thus, the natural cartilage is maintained at the regions of the acetabulum exposed to the greatest strain during normal joint articulation, such as the cartilage at the polar region of the acetabulum, and at a region spaced at least 30°, 40°, or 45° therefrom, for example. Additionally, the present invention allows for the natural femoral head to articulate against the repaired acetabulum. Thus, the need to undergo total hip arthroplasty is eliminated.

In one form thereof, the present invention provides, an acetabular implant for the treatment of a defect in a bearing surface of an acetabulum, the acetabular implant including: a base body; and an elongate articulation portion defining a longitudinal axis and a substantially spherical articular surface defined by a portion of a sphere that substantially mimics the shape of the acetabulum, the substantially spherical articular surface extending along the longitudinal axis of the elongate articulation portion over a first angle and extending perpendicular to the longitudinal axis of the elongate articulation portion over a second angle, wherein the first angle is less than 180 degrees and the second angle is less than 90 degrees.

In another form thereof, the present invention provides an acetabular implant for the treatment of a defect in a bearing surface of an acetabulum, the acetabular implant including a first member having a base body and a substantially circular articulation portion having a substantially spherical articular surface, the substantially spherical articular surface defined by a portion of a sphere that substantially mimics the shape of the acetabulum, the substantially spherical articular surface extending across the articulation portion over a first angle extending from a pole of the substantially spherical articular surface to a line on the substantially spherical articular surface, the line parallel to an equator of the substantially spherical articular surface, the first angle measured in a plane perpendicular to the equator of the substantially spherical articular surface, wherein the first angle is less than 60 degrees.

In yet another form thereof, the present invention provides an acetabular implant for the treatment of a defect in a bearing surface of an acetabulum, the acetabular implant including a ring having a polar edge, an equatorial edge, and a substantially spherical articular surface extending between the polar edge and the equatorial edge, the substantially spherical articular surface defined by a portion of a sphere that substantially mimics the shape of the acetabulum, the substantially spherical articular surface extending from the polar edge to the equatorial edge over a first angle, the first angle contained in a plane perpendicular to the equatorial edge, wherein the first angle is less than 80 degrees.

In yet another form thereof, the present invention provides a method of repairing a defect in a bearing surface of an acetabulum, the acetabulum having a pole and an equator, the method including the steps of: providing an acetabular implant; removing the defect in the bearing surface of the acetabulum; shaping the acetabulum to receive the acetabular implant; and implanting the acetabular implant at a position spaced from the pole of the acetabulum by a first angular distance measured along a portion of a sphere that substantially mimics the shape of the acetabulum, the first distance defined by an implantation angle extending from the pole of the acetabulum toward the equator of the acetabulum, the implantation angle measuring at least 30 degrees, wherein the acetabular implant is at least 30 degrees from the pole of the acetabulum.

In yet another form thereof, the present invention provides a method of repairing a defect in a bearing surface of an acetabulum, the method including the steps of: providing an acetabular implant, the acetabular implant including: a base body and an articulation portion having a substantially spherical articular surface, the substantially spherical articular surface defined by a portion of a sphere that substantially mimics the shape of the acetabulum, the substantially spherical articular surface extending across the articulation portion over a first angle, wherein the first angle is less than 180 degrees; removing the defect in the bearing surface of the acetabulum; shaping the acetabulum to receive the acetabular implant; and implanting the acetabular implant in the acetabular, whereby the substantially spherical articular surface of the acetabular implant is aligned with the bearing surface of the acetabulum to provide a substantially seamless transition therebetween.

### BRIEF DESCRIPTION OF THE DRAWINGS

FIG. 1 is a perspective view of a pelvis with an exemplary implant of the present invention positioned in the acetabulum;

FIG. 1A is a series of exemplary views of the pelvis of FIG. 1 depicting an area of damaged cartilage on an acetabulum;

FIG. 2 is a perspective view of an orthopedic component according to an exemplary embodiment;

FIG. 3 is a perspective view of an orthopedic component according to another exemplary embodiment;

FIG. 4 is a perspective view of an orthopedic component according to yet another exemplary embodiment;

FIG. 5 is a perspective view of an orthopedic component according to yet another exemplary embodiment;

FIG. 5a is an illustration of an orthopedic component according to yet another exemplary embodiment;

FIG. 6 is a perspective view of an orthopedic component according to yet another exemplary embodiment;

FIG. 6A is a perspective view of a pelvis depicting the orthopedic component of FIG. 6 implanted within an acetabulum;

FIG. 7 is an exploded, perspective view of a bone shaping instrument;

FIG. 8 is a perspective view of a pelvis depicting the orthopedic components of FIG. 9 implanted within an acetabulum;

FIG. 9 is a perspective of a plurality of orthopedic components according to another exemplary embodiment;

FIG. 10 is a perspective of one of the plurality of orthopedic components of FIG. 9;

FIG. 11 is a perspective of another of the plurality of orthopedic components of FIG. 9;

FIG. 12 is a perspective view of a pelvis depicting the orthopedic component of FIG. 13 implanted within an acetabulum; and

FIG. 13 is a perspective view of an orthopedic component according to another exemplary embodiment.

### DETAILED DESCRIPTION

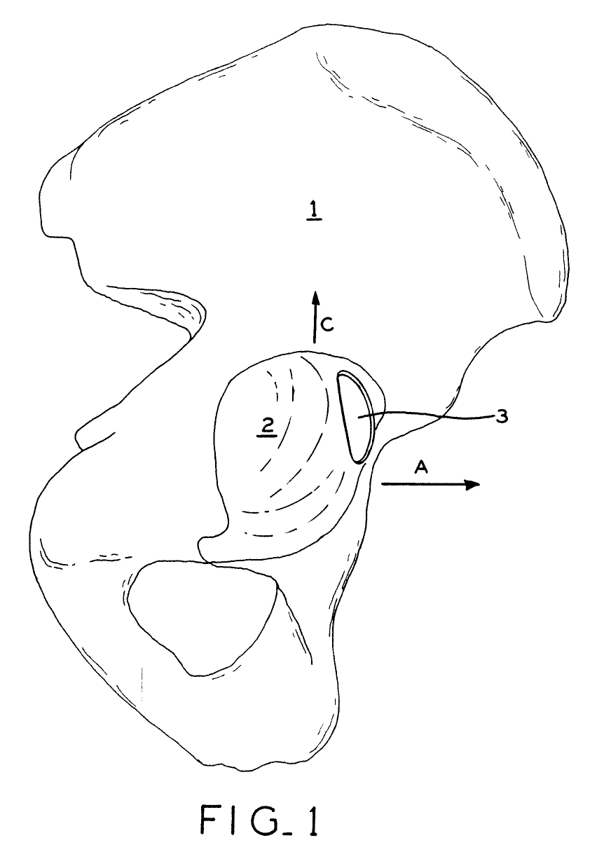

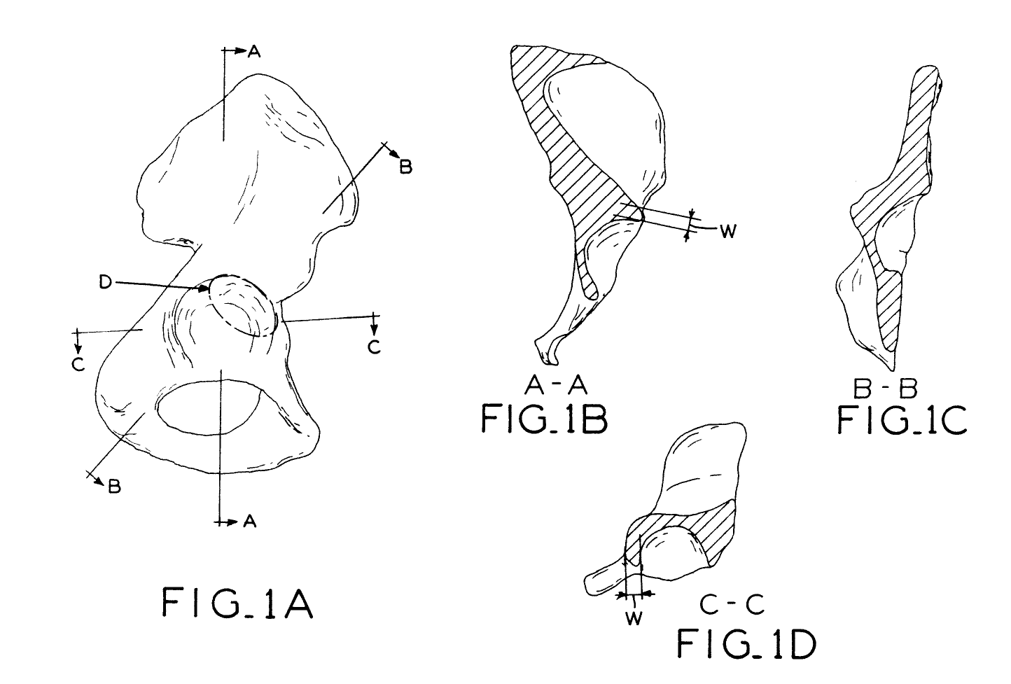

Referring to FIGS. 1 and 1A, pelvic bone 1 is shown including acetabulum 2, which forms the “joint socket” of the hip joint. In order to provide anatomical orientations to the Figures, the arrow “A” is used to denote the anterior direction and the arrow “C” is used to denote the cranial direction. In order to facilitate articulation of a femoral head within acetabulum 2, acetabulum 2 is substantially covered with articular cartilage. As discussed above, a portion of the articular cartilage may become damaged over time. Referring to FIG. 1A, a damaged portion of cartilage is shown at area D of acetabulum 2. As shown in FIG. 1, orthopedic implant 3 is positioned within a portion of acetabulum 2 to replace the cartilage at damaged area D (FIG. 1A) and substantially replicate the function of the same.

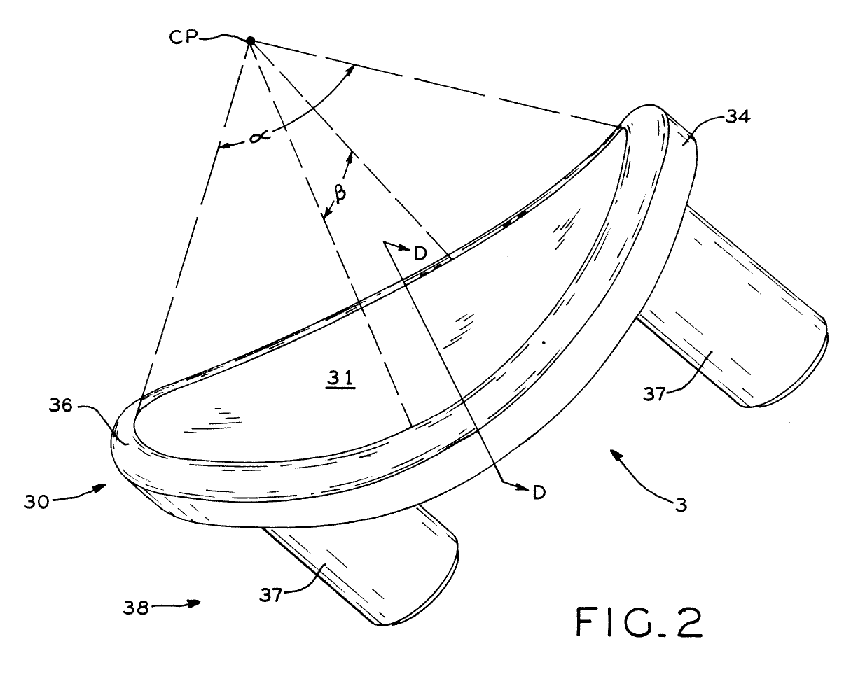

Referring to FIG. 2, orthopedic implant 3 is shown. Implant 3 includes articulation portion 30 having articular surface 31 and base body 38. In one exemplary embodiment, articulation portion 30 of orthopedic implant 3 has a cross-section taken in the direction of arrows D-D of FIG. 2 that is generally lens-shaped. In one exemplary embodiment, articular surface 31 of orthopedic implant 3 is a generally elliptical portion of a spherical surface. In another exemplary embodiment, articular surface 31 is a generally oval portion of a spherical surface.

As shown in FIG. 2, the angle of the substantially spherical surface defined by articular surface 31 extending in the direction of the longitudinal axis of implant 3, i.e., the long axis, of implant 3, covers less than 180°. Stated another way, angle α defined between opposing lines extending from the longitudinal extremes of articular surface 31 to centerpoint CP of spherical articular surface 31 is less than 180°. Thus, articular surface 31 extends in a longitudinal direction less than one-half of the distance around a sphere having a radius matching the radius of curvature of articular surface 31. In one exemplary embodiment, angle α is less than 90°. For example, angle α may be as small as 20°, 30°, 40°, or 50° or as large as 60°, 70°, 80°, or 90°. In one exemplary embodiment, angle α is less than 90° and greater than 45°.

Similarly, the angle of the substantially spherical surface defined by articular surface 31 extending in the direction perpendicular to the longitudinal axis of implant 3 covers less than 90°. Stated another way, angle β defined between lines extending from opposing extremes of articular surface 31 perpendicular to the longitudinal axis of implant 3 to centerpoint CP of spherical articular surface 31 is less than 90°. Thus, articular surface 31 extends in a direction perpendicular to the longitudinal axis of implant 3 for a distance less than one-quarter of the distance around a sphere having a radius matching the radius of curvature of articular surface 31. In one exemplary embodiment, angle β is less than 45°. For example, angle β may be as small as 10°, 15°, 20°, or 25° or as large as 30°, 35°, 40°, or 45°. Referring to FIG. 2, a plane containing angle β is perpendicular to a plane containing angle β.

As shown in FIG. 2, upper surface 36 of articulation portion 30 extending between periphery 34 of articulation portion 30 and articular surface 31 may be curved and/or chamfered. Additionally, extending from articulation portion 30 are a pair of anchorage members 37 which combine to define base body 38. While described and depicted herein as having a pair of anchorage members 37, any number of anchorage members 37, including a single anchorage member 37, may be provided. As shown in FIG. 2, anchorage members 37 have a substantially cylindrical shape. In one exemplary embodiment, implant 3 and, thus, both articulation portion 30 and base body 38 are formed from the same material, e.g., PEEK, PEKK. Thus, both articulation surface 31 defined by articulation portion 30 and anchorage members 37 that define base body 38 are formed from the same material. In one exemplary embodiment, implant 3 is formed from one of a hydrogel, a polyethylene (PE), a polyurethane (PU), a polycarbonate urethane (PCU), a polyvinyl alcohol (PVA), or a material of pyrolytic carbon. If implant 3 is formed from a polyethylene, for example, bone cement may be used to secure anchorage members 37 in apertures formed in the acetabulum, as described in detail below.

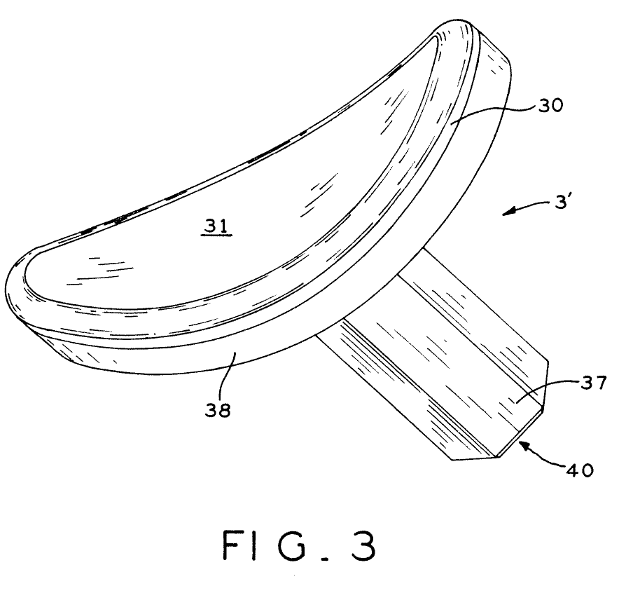

Referring to FIG. 3, another exemplary embodiment of implant 3 is depicted as implant 3′. Implant 3′ has several features which are identical to implant 3 of FIGS. 1 and 2, discussed above, and identical reference numerals have been used to identify identical or substantially identical features therebetween. Implant 3′ includes articulation portion 30′ defining articular surface 31 and base body 38. Base body 38 extends substantially entirely along the underside of articulation portion 30′ to provide support to articulation portion 30′. In one exemplary embodiment, articulation portion 30′ is connected to base body 38 to rigidly secure articulation portion 30′ to base body 38. In one exemplary embodiment, base body 38 is formed from a plurality of layers of differently orientated titanium wires or titanium mesh, which can also have different diameters. In another exemplary embodiment, articulation portion 30 is a polymer, such as polyethylene, and base body 38 is a highly porous biomaterial. In an exemplary embodiment, base body 38 is made from titanium or tantalum, which forms a porous metal structure such as CoCr beads, woven or nonwoven structures, orderly oriented wire meshes such as “Sulmesh” available from ZIMMER GMBH or porous metal structures like “Trabecular Metal”, which is further described hereinbelow. These metal structures can also be formed by applying textile techniques (e.g., weaving or knitting) using different kinds and mixtures of metal and/or non-metallic wires. In another embodiment base body 38 can be designed to be flexible to allow geometrical adaptation of the implant to the defect morphology.

A highly porous biomaterial may have a porosity as low as 55, 65, or 75 percent or as high as 80, 85, or 90 percent. An example of such a material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, the entire disclosure of which is expressly incorporated herein by reference. In addition to tantalum, other metals such as niobium, or alloys of tantalum and niobium with one another or with other metals may also be used.

Generally, the porous tantalum structure includes a large plurality of ligaments defining open spaces therebetween, with each ligament generally including a carbon core covered by a thin film of metal such as tantalum, for example. The open spaces between the ligaments form a matrix of continuous channels having no dead ends, such that growth of cancellous bone through the porous tantalum structure is uninhibited. The porous tantalum may include up to 75%-85% or more void space therein. Thus, porous tantalum is a lightweight, strong porous structure which is substantially uniform and consistent in composition, and closely resembles the structure of natural cancellous bone, thereby providing a matrix into which cancellous bone may grow to anchor implant 3′ in the surrounding bone of the pelvis.

In this embodiment, articulation portion 30 may be molded onto base body 38. For example, implant 3′ may be formed according to the teachings of co-pending U.S. patent application Ser. No. 11/095,217, entitled HYDROGEL IMPLANT, filed on Mar. 31, 2005, the entire disclosure of which is expressly incorporated by reference herein. By utilizing a highly porous biomaterial, e.g., a material formed using Trabecular Metal™ technology, a metal having a porous coating, or a metal having a knurled surface, to form base body 38, the attachment of articulation portion 30 allows for some of the biocompatible polymer forming articulation portion 30 to be received within the pores of base body 38. This interaction creates a rigid, mechanical bond between articulation portion 30 and base body 38 to fixedly attach the two layers together, which allows for the transfer of the forces between articulation portion 30 and base body 38 that are encountered during the loading of the joint.

Referring again to FIG. 3, base body 38 further includes anchoring member 37 extending therefrom. Thus, by forming base body 38 from a highly porous biomaterial, for example, anchoring member 37 is, as an integral component of base body 38, formed from the same material. In one exemplary embodiment, anchoring member 37 has a substantially hexagonal cross-section taken perpendicular to the longitudinal axis of anchoring member 37. Additionally, to facilitate insertion of anchoring member 37 into the acetabulum, as described in detail below, end 40 of anchoring member 37 may be curved and/or chamfered. In one exemplary embodiment, anchoring member 37 is coated with a bone ingrowth material, such as hydroxyl apatite. By forming base body 38 and, correspondingly, anchoring member 37 from a highly porous biomaterial and/or applying a bone ingrowth coating thereto, implant 3′ may be particularly suitable for cement-free implantation.

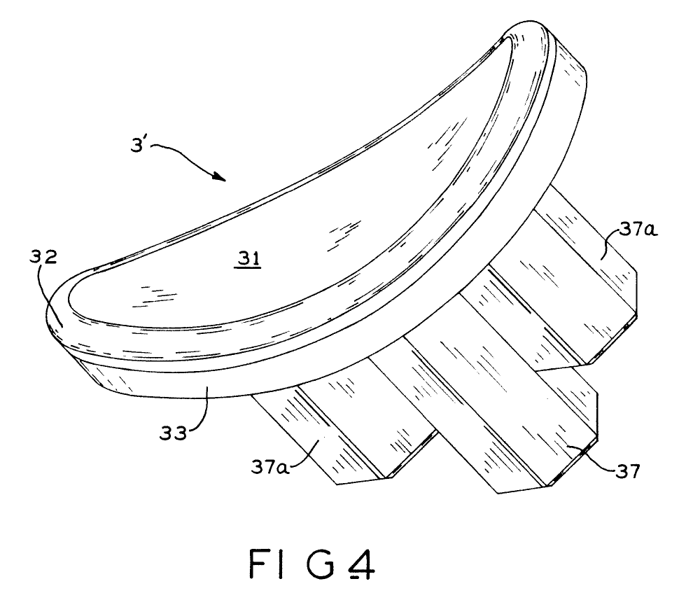

Referring to FIG. 4, implant 3′ is shown with a plurality of anchoring members 37, 37a. Thus, instead of a single anchoring member 37, implant 3′ includes a plurality of differently size anchoring members 37, 37a extending therefrom. In this embodiment, anchoring members 37a are slightly smaller than anchoring member 37. Specifically, anchoring members 37a do not extend as far from the convex undersurface of implant 3′ as does anchoring member 37. In alternative embodiments, one or both anchoring members 37a may be smaller in cross-section that anchoring member 37. Anchoring members 37, 37a provide for improved fixation of implant 3′ to a patient's acetabulum and help prevent rotation of implant 3′ once implanted.

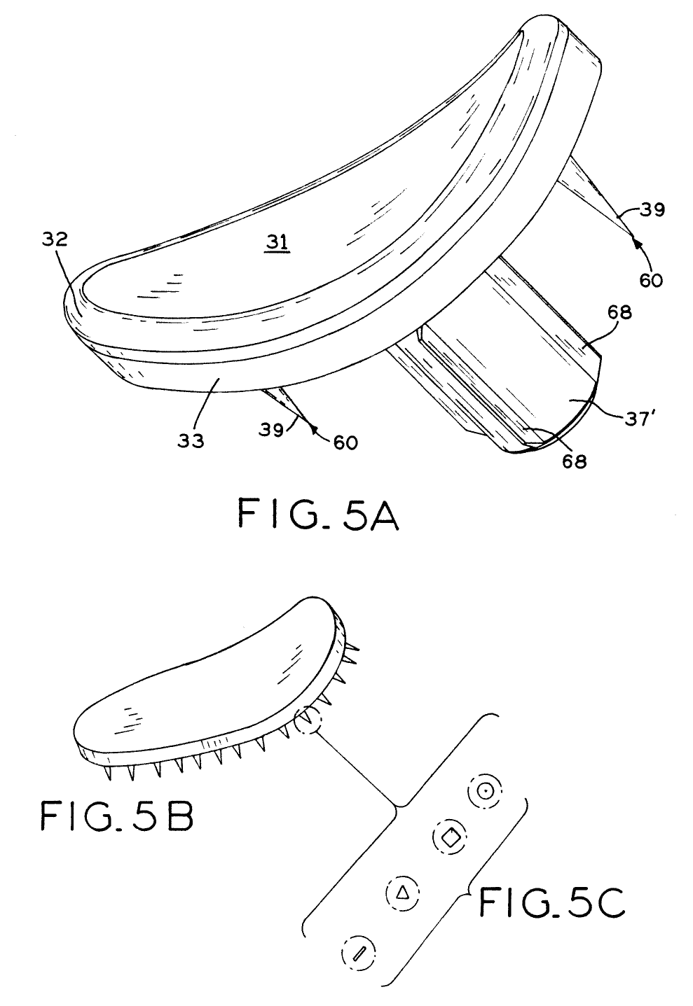

Referring to FIG. 5, implant 3′ is shown with anchoring member 37′ and pins 39 according to another exemplary embodiment. Anchoring member 37′ includes a substantially cylindrical shaped core with a plurality of fins 68 extending radially outwardly therefrom. Pins 39 have a substantially cone shape that tapers to form a point at end 60. In another exemplary embodiment, fins 68 are substantially flat, planar structures that also taper to a point. Fins 68 and pins 39 facilitate the primary anchorage of implant 3′ to bone and also help to prevent rotation of implant 3′. Additionally, end 60 of anchoring member 37′ may be curved and/or chamfered to facilitate the insertion of anchoring member 37′ into the acetabulum.

In the embodiment shown in FIG. 5a the fixation area is comprised of an array of metal and/or non-metal spikes. Those spikes can be used with a flexible or nonflexible implant to provide primary fixation via the spikes and secondary fixation by allowing osseo integration of the spike into underlying bone stock. Such a spike array in combination with a flexible implant may also be advantageous to allow intra-operative adaptation of the implant shape to the defect morphology/size by using provided cutting instruments. The cross section of the spike can be for example round or rectangular or blade-shaped and the surface is roughened between Ra=1 micron to 10 micron.

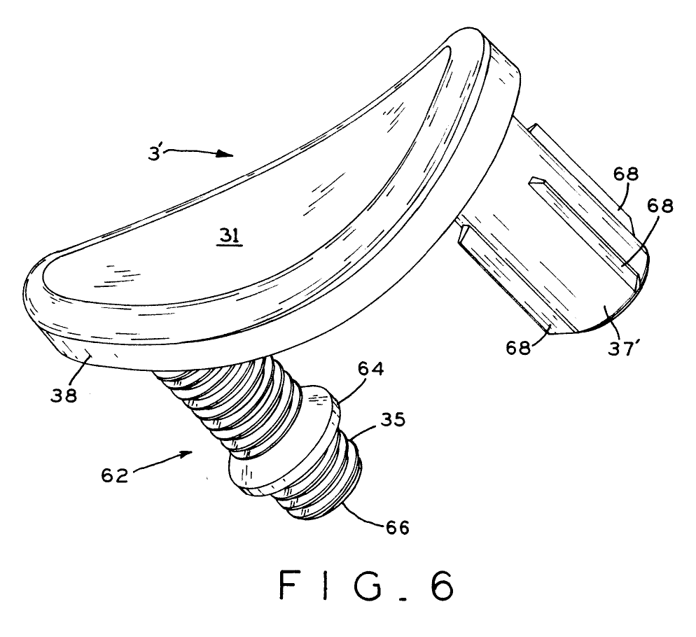

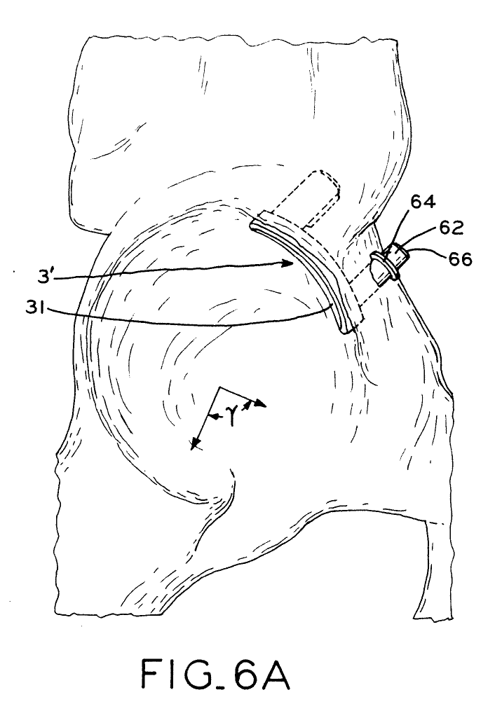

Referring to FIG. 6, implant 3′ is shown with anchoring members 37′, 62 according to another exemplary embodiment. Anchoring member 62 has a substantially cylindrical shape and includes thread 35 extending radially outwardly therefrom. Nut 64 is configured for threaded receipt on anchoring member 62. Referring to FIG. 6A, implant 3′ is shown with anchoring member 62 extending entirely through the bone of the pelvis, such that end 66 of anchoring member 62 extends outwardly from the pelvic bone into the patient's body. By positioning anchoring member 62 in an anterior direction, as shown in FIG. 6, the depth of bone stock of the pelvis is smaller that at other areas around the acetabulum, as shown in Fig. 1A having width W. With anchoring member 62 positioned through the bone of the pelvis, nut 64 may then be threaded onto anchoring member 62 to secure implant 3′ in position. Additionally, with implant 3′ in this position, anchoring member 37′ extends into an area of greater bone stock of the pelvis (such that anchoring member 37′ does not extend from the pelvic bone opposite the acetabulum) and further facilitates the retention of implant 3′ in position.

Referring to FIG. 6, nut 64 includes an upper surface facing the convex underside of implant 3′. The upper surface of nut 64 tapers inward toward anchoring member 62. Nut 64 can include any feature to facilitate connection to a driver, such as an external hex extending from the undersurface of nut 64. Implants in accordance with the present invention may include any combination of the various anchoring features described in this document.

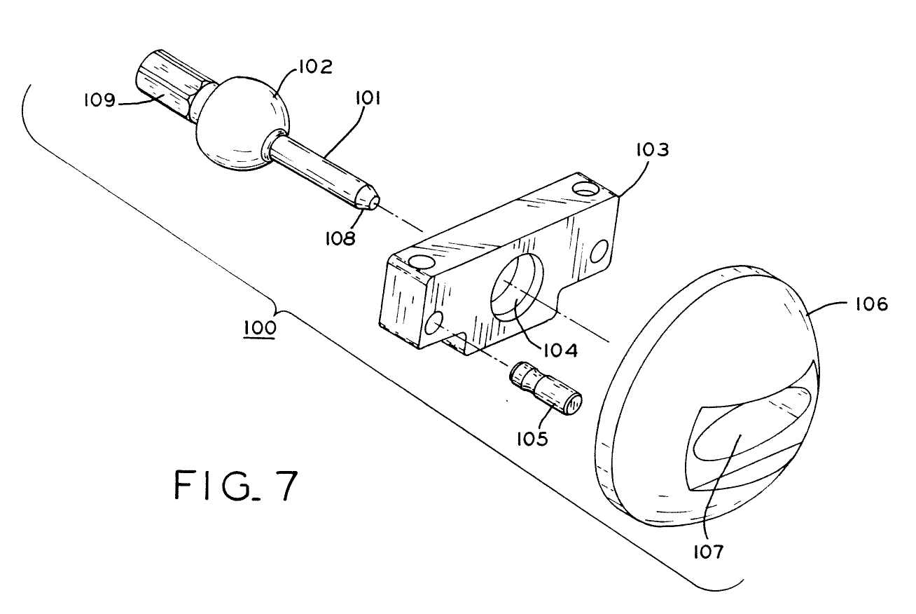

In order to facilitate the implantation of implant 3, 3′, an incision may be made in the area of the patient's acetabulum. The damaged portions of articular cartilage are then removed and the acetabulum prepared for the receipt of implant 3, 3′. Specifically, in order to prepare the acetabulum, instrument 100, shown in FIG. 7, may be used. Instrument 100 facilitates the shaping of the acetabulum for the insertion of implant 3, 3′. Instrument 100 includes a first component having shaft 101 extending from spherical guide member 102 substantially colinearly with proximal actuation end 109. Distal end 108 of shaft 101 includes a connection device, such as a tapered cone, for coupling to working head 105, for example. In one exemplary embodiment, working head 105 forms a reamer. Distal end 108 of shaft 101 is positioned to extend through guide member 103. Guide member 103 includes concavely spherical guide surface 104 that has a shape substantially similar to a portion of spherical guide member 102, such that spherical guide member 102 is free to smoothly rotate against guide surface 104, while being thereby held in place. To assemble these components, spherical guide member is snapped into spherical guide surface 104.

Guide member 103 is then positioned against machining mask 106. Machining mask 106 has the basic shape of a spherical cap and fits into the acetabulum. Machining mask 106 is inserted in the acetabulum such that opening 107 comes to lie above the defect in the cartilage of the acetabulum. With guide member 103 positioned against machining mask 106, shaft 101 and working head 105 extend through guide member 103 and opening 107 in machining mask 106. Opening 107 in machining mask 106 is dimensioned such that actuation of shaft 101 and guide member 102 about guide surface 104 is restricted by shaft 101 contacting the outer periphery of opening 107. Thus, working head 105 is only allowed to move in a shape that substantially replicates the shape of implant 3, 3′. Thus, a surface corresponding to implant 3, 3′ is machined into the acetabulum. Specifically, the acetabulum is machined to provide a congruence between the articular cartilage of the acetabulum and articular surface 31 of implant 3, 3′. This congruence provides a substantially seamless transition between articular surface 31 and the articular cartilage of the acetabulum. To provide this seamless transition, working head 105 protrudes a distance from the spherically convex outer surface of machining mask 106 that is equal to the distance between the spherically concave bearing surface of implant 3, 3′ and the spherically convex bone contacting surface of implant 3, 3′.

Once a surface of the acetabulum is properly machined, bores may be drilled in the acetabulum to receive anchoring members 37, 37′, 62. In one exemplary embodiment, the bores are sized to provide a press-fit of anchoring members 37, 37′ therein. In this embodiment, implant 3, 3′ may be secure to the acetabulum without the use of bone cement. In another exemplary embodiment, the bores may be formed in the acetabulum for the receipt of both anchoring members 37, 37′ and bone cement. In this embodiment, the securement of implant 3, 3′ to the bone is further enhanced by the use of the bone cement.

Advantageously, by using implants 3, 3′, the articular cartilage at the polar region of the acetabulum may be retained. For example, referring to FIG. 6A, implant 3′ is positioned toward the equator of the acetabulum. Thus, the natural cartilage is maintained at the regions of the acetabulum exposed to the greatest strain during normal joint articulation, such as the cartilage at the polar region of the acetabulum. In one exemplary embodiment, implants 3, 3′ are implanted at a distance spaced from the pole or deepest point of the acetabulum by an implantation angle γ. Implantation angle γ is an angle measured along the spherical articulation surface of the acetabulum. Angle γ is contained in a plane containing the pole of the articulation surface of the acetabulum and being perpendicular to an equator of an acetabulum. For example, a 30° implantation angle corresponds to the 60° parallel or line of latitude, i.e., a line on the spherical articulation surface of the acetabulum that is parallel to the equator of the acetabulum and spaced 60° from the equator toward the pole of the acetabulum. For example, as shown in FIG. 6A, implant 3′ is spaced from the polar axis of the acetabulum by an implantation angle of at least 30°. In exemplary embodiment, implantation angle γ may be at least 35°, 40°, or 45°, for example.

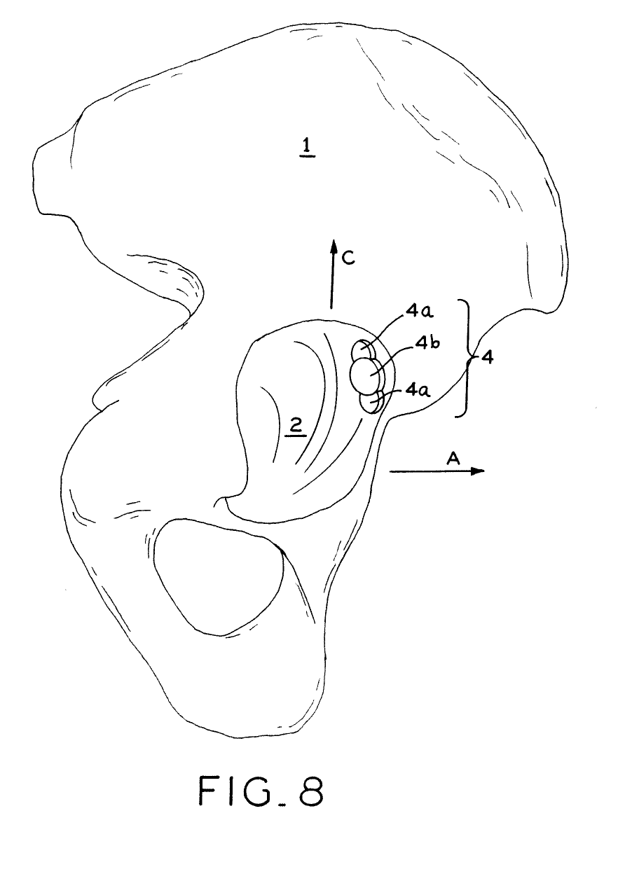

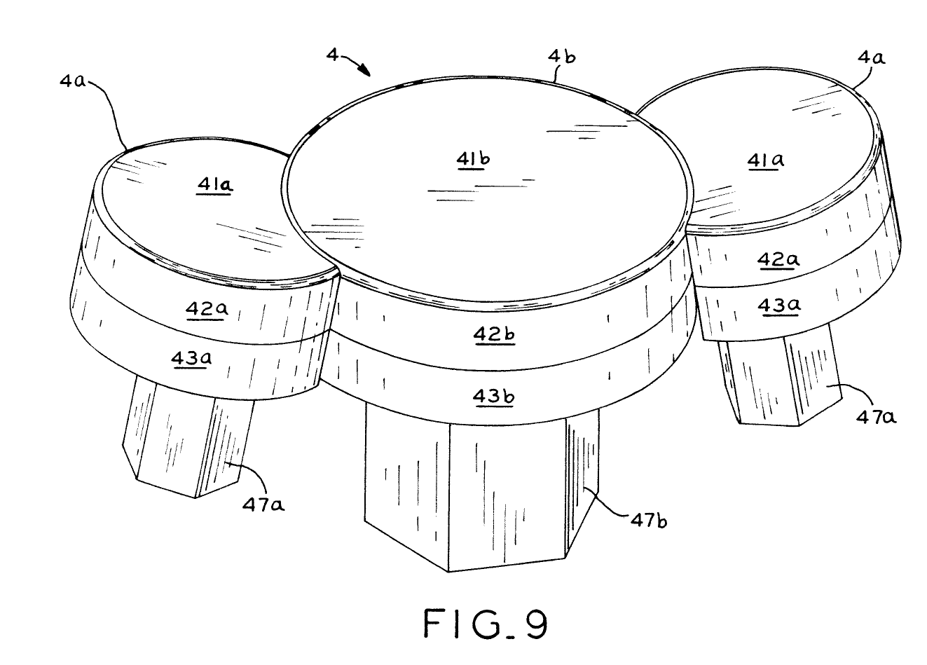

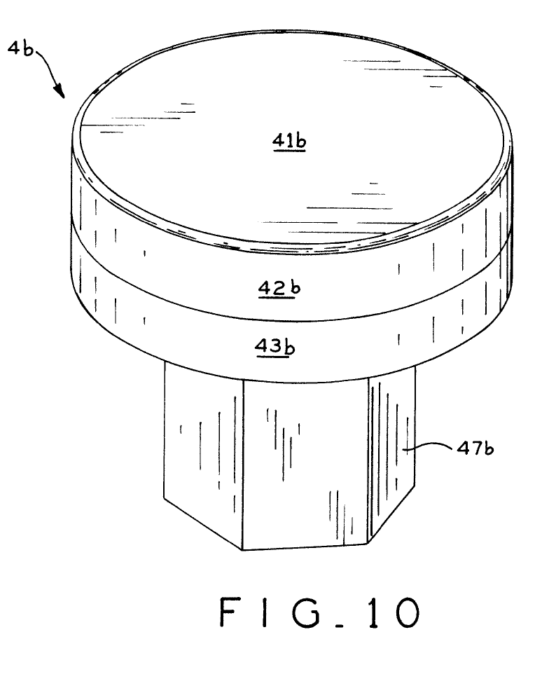

Referring to FIGS. 8-11, another exemplary implant design is depicted as implant 4. Implant 4 has several features that are identical or substantially identical to corresponding features of implant 3, 3′ and identical reference numerals have been used to identify corresponding features therebetween. Implant 4 includes mushroom-shaped members 4a, 4b having articulation portions 42a, 42b including articular surfaces 41a, 41b that are substantially circular in shape and base bodies 43a, 43b, respectively. Base bodies 43a, 43b of implant 4 are sized for receipt within the acetabulum and include anchoring members 47a, 47b extending therefrom. In one exemplary embodiment, anchoring members 47a, 47b have a substantially hexagonal cross-section taken perpendicular to the longitudinal axis of anchoring members 47a, 47b. With respect to articular surfaces 41a, 41b, the angle of the substantially spherical surface defined by articular surfaces 41a, 41b covers less than 60°. Stated another way, an angle defined between lines extending from opposing sides of surfaces 41a, 41b to centerpoint of a sphere having a radius matching the radius of curvature of articular surfaces 41a, 41b and positioned in a plane containing the opposing points and the center point of the sphere is less than 60°. When the spherical surface covers less than 60° as defined above, the spherical surface corresponds to the cap of a sphere from it's pole to a 30° parallel or line of latitude. In one exemplary embodiment, the angle is less than 45°. In other exemplary embodiments, the angle may be as small as 5°, 10°, 15°, or 20° or as large as 30°, 40°, 50°, or 60°.

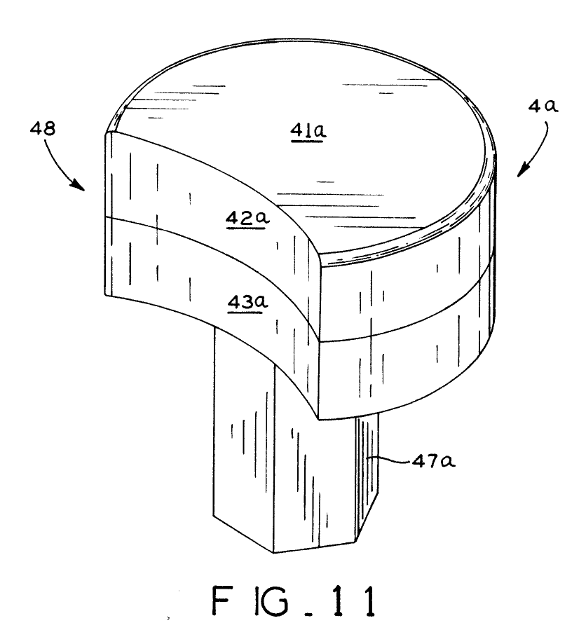

Referring to FIG. 11, members 4a also include cut-outs 48 that have a curvature substantially similar to the curvature of articulation portion 41b of member 4b. As a result of cut-outs 48, articulation portion 41b of members 4a have a substantially circular, crescent shape. Thus, members 4a may be positioned adjacent to members 4b and aligned therewith to provide a substantially seamless transition therebetween. By using a plurality of members 4a, 4b a defect that has unique shape may be treated. For example, the defect may be substantially larger or smaller in size than the defected treated by implant 3, 3′. By simply altering the number of members 4a, 4b that are used, the size of implant 4 is effectively changed to better correlate to the defect size. Additionally, implant 4 may be implanted in an acetabulum using the technique described in detail above. However, in order to prepare the proper shape on the acetabular surface, another machine mask, such as a machining mask substantially similar to machining mask 106, having an opening shaped to substantially correlate to the desired shape of implant 4 may be used. Alternatively, machining mask 106 may be repositioned for additional reaming after initial reaming is complete.

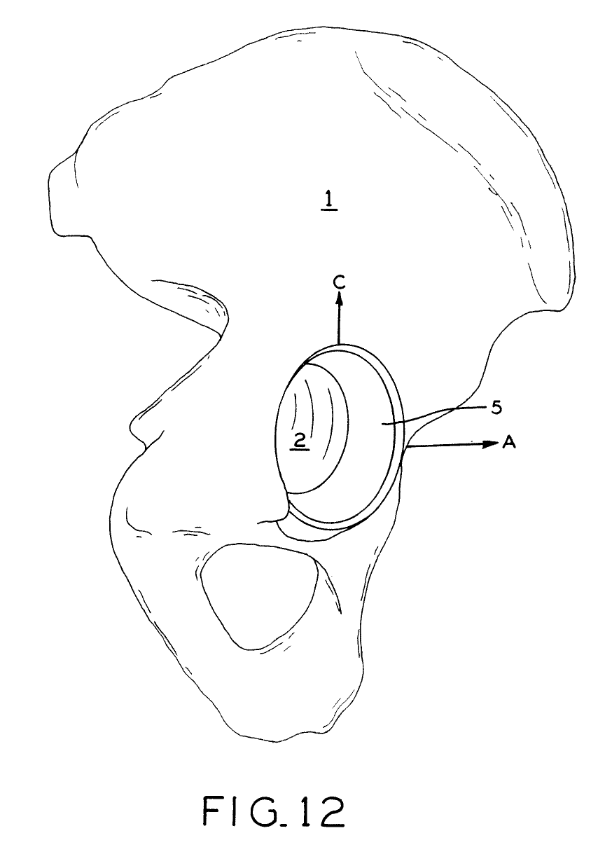

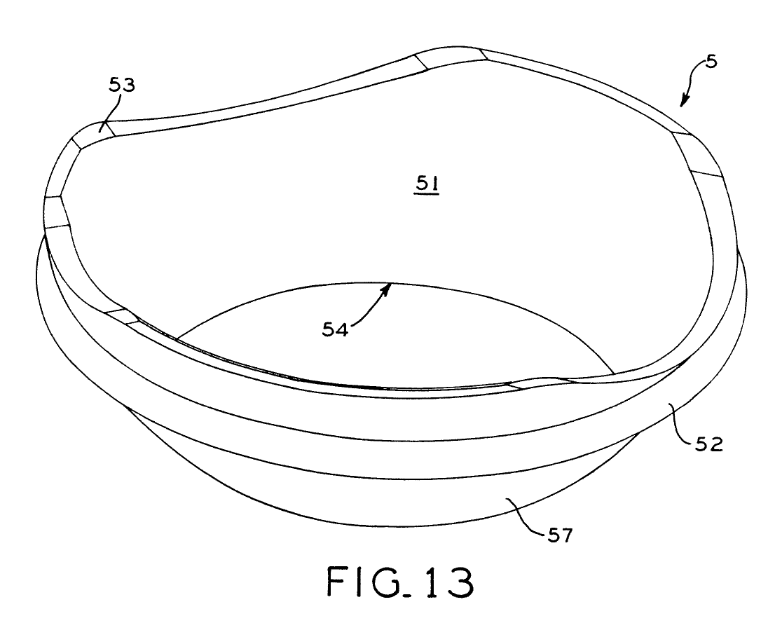

Referring to FIGS. 12 and 13, another exemplary implant in the form of elastic ring 5 is shown. Elastic ring 5 is configured to replace a larger portion of the acetabulum than implants 3, 3′, 4 and may be inserted to extend around an inner portion of the acetabulum. As shown in FIG. 13, elastic ring 5 has the shape of a spherical cap which is cut off at its pole. Ring 5 also includes a substantially concave, spherical articulation surface 51. Edges 54, 53 extends around ring 5 adjacent the pole of ring 5 and the equator of ring 5, respectively. Additionally, edge 54 is cut along a substantially flat plane. In contrast, edge 53 is contoured to substantially mimic the edge of a patient's acetabulum. Thus, edge 54 substantially follows the outer edge of the acetabulum as accurately as possible without allowing for edge 54 to project excessively over the outer edge of the acetabulum. By substantially preventing edge 54 from projecting over the outer edge of the acetabulum, impingement of the femoral neck on edge 54 of ring 5 is substantially prevented.

Extending around the outer surface 57 of ring 5 is bead 52. Bead 52 is configured to engage a groove that is machined into the acetabulum during implantation of ring 5. Specifically, by press-fitting ring 5 into the acetabulum, bead 52 may form a snap-fit connection with the groove in the acetabulum. The interaction of bead 52 with the corresponding groove formed in the acetabulum prevents ring 5 from separating from the acetabulum and facilitates the retention of ring 5 in position.

As indicated above, ring 5 has a substantially concave, spherical articulation surface 51. The angle of articulation surface 51 between edge 53 and edge 54 of ring 5 covers less than 80°. Stated another way, an angle defined between lines extending from opposing edges 53, 54 of articular surface 51 to a centerpoint of a sphere having a radius matching the radius of curvature of articular surface 51 and positioned in a plane containing the center point of the sphere and a pole of the sphere is less than 80°. In one exemplary embodiment, the angle is less than 60°. For example, the angle may be as small as 30°, 35°, 40°, or 45° or as large as 50°, 60°, 70°, or 80°.

In one exemplary embodiment, the opening half-angle of the cap which is “cut-off” from the implant at the polar side and therefore defines the polar opening in which the natural cartilage is maintained is at least 10°. In exemplary embodiments, the half-angle may be as small as 30°, 35°, 40°, or 45° or as large as 50°, 55°, 60°, or 65°.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An acetabular implant for the treatment of a defect in a bearing surface of an acetabulum, the acetabular implant comprising:
a base body; and
an elongate articulation portion defining a longitudinal axis and a substantially spherical articular surface defined by a portion of a sphere that substantially mimics the shape of the acetabulum, said substantially spherical articular surface extending along said longitudinal axis of said elongate articulation portion over a first angle and extending perpendicular to said longitudinal axis of said elongate articulation portion over a second angle, wherein said first angle is less than 180 degrees and said second angle is less than 90 degrees.

2. The acetabular implant of claim 1, wherein said first angle is less than 90 degrees and said second angle is less than 45 degrees.

3. The acetabular implant of claim 1, wherein said base body further comprises an anchoring member.

4. The acetabular implant of claim 3, wherein said anchoring member has a substantially cylindrical cross-section taken perpendicular to a longitudinal axis of said anchoring member.

5. The acetabular implant of claim 4, wherein said anchoring member further comprises a fin extending radially outwardly therefrom.

6. The acetabular implant of claim 3, wherein said anchoring member has a substantially hexagonal cross-section taken perpendicular to a longitudinal axis of said anchoring member.

7. The acetabular implant of claim 3, wherein said anchoring member comprises a threaded stud, said acetabular implant further comprising a nut threadably connectable to said threaded stud.

8. An acetabular implant for the treatment of a defect in a bearing surface of an acetabulum, the acetabular implant comprising:
a first member having a base body and a substantially circular articulation portion having a substantially spherical articular surface, said substantially spherical articular surface defined by a portion of a sphere that substantially mimics the shape of the acetabulum, said substantially spherical articular surface extending across said articulation portion over a first angle extending from a pole of said substantially spherical articular surface to a line on said substantially spherical articular surface, said line parallel to an equator of said substantially spherical articular surface, said first angle measured in a plane perpendicular to the equator of said substantially spherical articular surface, wherein said first angle is less than 60 degrees.

9. The acetabular implant of claim 8, wherein said first angle is less than 45 degrees.

10. The acetabular implant of claim 8, further comprising a second member, said second member having a base body and a substantially crescent shaped articulation portion, said crescent shaped articulation portion at least partially defined by a cut-out, said articulation portion having a substantially spherical articular surface, said substantially articular surface defined by a portion of a sphere that substantially mimics the shape of the acetabulum, said substantially spherical articular surface extending across said articulation portion over a second angle, extending from a pole of said substantially spherical articular surface to a line on said substantially spherical articular surface, said line parallel to an equator of said substantially spherical articular surface, said second angle measured in a plane perpendicular to the equator of said substantially spherical articular surface wherein said second angle is less than 60 degrees.

11. The acetabular implant of claim 10, wherein one of said first angle and said second angle comprise less than 45 degrees.

12. The acetabular implant of claim 10, wherein said cut-out conforms to an exterior surface of said first member, whereby a surface defining said cutout can be positioned flush within the exterior surface of said first member.

13. An acetabular implant for the treatment of a defect in a bearing surface of an acetabulum, the acetabular implant comprising:
a ring having a polar edge, an equatorial edge, and a substantially spherical articular surface extending between said polar edge and said equatorial edge, said substantially spherical articular surface defined by a portion of a sphere that substantially mimics the shape of the acetabulum, said substantially spherical articular surface extending from said polar edge to said equatorial edge over a first angle, said first angle contained in a plane perpendicular to said equatorial edge, wherein said first angle is less than 80 degrees.

14. The acetabular implant of claim 13, wherein said first angle is less than 60 degrees.

15. The acetabular implant of claim 13, wherein said ring further comprises a bead extending radially outwardly therefrom, whereby said bead facilitates the retention of said elastic ring within the acetabulum.

16. A method of repairing a defect in a bearing surface of an acetabulum, the method comprising the steps of:
providing an acetabular implant, the acetabular implant comprising:
a base body and an articulation portion having a substantially spherical articular surface, the substantially spherical articular surface defined by a portion of a sphere that substantially mimics the shape of the acetabulum, the substantially spherical articular surface extending across the articulation portion over a first angle, wherein the first angle is less than 180 degrees;
removing the defect in the bearing surface of the acetabulum;
shaping the acetabulum to receive the acetabular implant; and
implanting the acetabular implant in the acetabular, whereby the substantially spherical articular surface of the acetabular implant is aligned with the bearing surface of the acetabulum to provide a substantially seamless transition therebetween.

17. The method of repairing a defect of claim 16, further comprising the step of drilling a bore into the acetabulum.

18. The method of repairing a defect of claim 16, wherein the first angle is less than 90 degrees.

19. The method of repairing a defect of claim 16, wherein the first angle is less than 45 degrees.

20. A method of repairing a defect in a bearing surface of an acetabulum, the acetabulum having a pole and an equator, the method comprising the steps of:
providing an acetabular implant;
removing the defect in the bearing surface of the acetabulum;
shaping the acetabulum to receive the acetabular implant; and
implanting the acetabular implant at a position spaced from the pole of the acetabulum by a first angular distance measured along a portion of a sphere that substantially mimics the shape of the acetabulum, the first distance defined by an implantation angle extending from the pole of the acetabulum toward the equator of the acetabulum, the implantation angle measuring at least 30 degrees, wherein the acetabular implant is at least 30 degrees from the pole of the acetabulum.

21. The method of claim 20, wherein the implantation angle is at least 40 degrees wherein the acetabular implant is at least 40 degrees from the pole of the acetabulum.

22. The method of claim 20, wherein the implantation angle is at least 60 degrees wherein the acetabular implant is at least 60 degrees from the pole of the acetabulum.

23. The method of claim 20, wherein the step of providing an acetabular implant further comprises providing an acetabular implant having a polar edge, an equatorial edge, and a substantially spherical articular surface extending between the polar edge and the equatorial edge, the substantially spherical articular surface defined by a portion of a sphere that substantially mimics the shape of the acetabulum, the substantially spherical articular surface extending from the polar edge to the equatorial edge over a first angle, wherein the first angle is less than 80 degrees.
